(19) United States
(12) Patent Application Publication
(10) Pub. No.: US 2023/0398041 A1
(43) Pub. Date: Dec. 14, 2023

(54) SOLID COMPOSITION COMPRISING THE COMBINATION OF AT LEAST TWO PARTICULAR ANIONIC SURFACTANTS

(71) Applicant: L'Oreal, Paris (FR)

(72) Inventors: Marion CRUVEILHIER, Saint Ouen (FR); Stéphanie FRANGI, Saint Ouen (FR); Géraldine FACK, Saint Ouen (FR)

(73) Assignee: L'Oréal, Paris (FR)

(21) Appl. No.: 18/044,916

(22) PCT Filed: Sep. 10, 2021
(86) PCT No.: PCT/EP2021/074937

§ 371 Date: Mar. 10, 2023

(30) Foreign Application Priority Data
Sep. 10, 2020 (FR) FR2009166

(51) Int. Cl. A61K 8/02; A61Q 5/02; A61Q 5/12; A61K 8/46

(52) U.S. Cl. CPC A61K 8/0216; A61Q 5/02; A61Q 5/12; A61K 8/463; A61K 2800/5426; A61K 2800/596

(57) ABSTRACT

The present invention relates to a solid composition comprising: a) one or more anionic surfactants of sulfate type, present in a total amount ranging from 10% to 35% by weight, relative to the total weight of the composition; b) one or more anionic surfactants of sulfonate type; c) one or more cationic polymers; and d) one or more (poly)glycerol esters.

The present invention relates to a solid composition intended in particular for washing keratin fibres, in particular human keratin fibres such as the hair, and which comprises a particular combination of at least two anionic surfactants, one of which is of sulfate type and the other of which is of sulfonate type, of at least one cationic polymer and of at least one (poly)glycerol ester.

The invention also relates to a cosmetic process for treating keratin fibres, in particular human keratin fibres such as the hair, using said solid composition.

The invention also relates to the use of said solid composition for washing and/or conditioning keratin fibres, in particular human keratin fibres such as the hair.

In the field of hair hygiene, products for washing keratin fibres are generally intended for cleansing said fibres while at the same time giving them good cosmetic properties. Conventional products, such as shampoos, are usually in the form of a more or less thickened liquid. Because of their liquid texture, these products can however have various drawbacks, and in particular can prove to be difficult to measure out.

Indeed, the more liquid they are, the more they have a tendency to run through the fingers, making them difficult to measure out and causing wastage. These products can also leak out of their packaging, which can be annoying for the consumer when these products come into contact with clothing or objects, for example during travel.

In order to modify the texture of these products, and to make it in particular more compact, thickeners are generally used. However, the addition of these compounds often takes place to the detriment of the cosmetic effects of the compositions. Moreover, the use of these thicker compositions requires a lot of rinsing water in order to remove the surplus product on the fibres. In point of fact, in many countries where access to water is restricted, the rinsing time and consequently the amount of water required to properly rinse off the product are key indicators of the working qualities of a composition.

In order to overcome some of these problems, new solid cosmetic formulations, in particular shampoos in the form of solid granules or powder, have been developed. However, these new formulations are not always entirely satisfactory. Those which are in the form of a free powder can in fact pose problems in terms of volatility, grasping and/or measuring out, while those which are in the form of aggregates, such as granules for example, can have a tendency to disaggregate or disintegrate with difficulty in the presence of water. Thus, the latter do not always make it possible to obtain a rapid foam initiation and/or a satisfactory abundance of foam, unfavourably impacting their use and their spreading on keratin fibres. They can also be difficult to remove during rinsing and even sometimes leave residues on the fibres that are unpleasant for the consumer.

Thus, there is a real need to provide a composition in solid form which has an improved environment profile, that is to say requiring little water throughout its use. The composition must not only have good foaming properties, in particular in terms of foam initiation, abundance, texture and density, but it must also be quick to rinse off while at the same time leaving as few residues as possible on the keratin fibres. Moreover, the composition must be pleasant to use without being tacky, nor must it leave a tacky effect on the keratin fibres.

The composition must also have a good detergent power while at the same time conferring satisfactory cosmetic properties, in particular in terms of manageability, feel, smoothness, softness, sheen and disentangling.

It has now been discovered that a solid composition comprising a particular combination of at least two anionic surfactants, one of which is of sulfate type and the other of which is of sulfonate type, of at least one cationic polymer and of at least one (poly)glycerol ester makes it possible to achieve the objectives set out above, and in particular to provide a composition in solid form which allies a good detergent power with improved foam properties, without however requiring large amounts of water, and confers good cosmetic properties, in particular in terms of manageability, feel, smoothness, softness, sheen and disentangling.

One subject of the present invention is thus a solid composition comprising:
a) one or more anionic surfactants of sulfate type, present in a total amount ranging from 10% to 35% by weight, relative to the total weight of the composition;
b) one or more anionic surfactants of sulfonate type;
c) one or more cationic polymers;
d) one or more (poly)glycerol esters.

The particular combination of the compounds of the invention makes it possible to obtain a compact solid composition that is easy to handle and to measure out. The composition may therefore be in the form of a bar or a stick, which form is particularly advantageous for example during travel or when practising a sport (lighter bags, limitation of the risks of leaking, reduction of waste).

This composition also solubilizes rapidly on contact with water and makes it possible to easily and rapidly obtain a firm, creamy and abundant foam of quality comparable to the foam obtained with a conventional liquid shampoo composition. This foam can subsequently be applied easily and uniformly to the keratin fibres.

Moreover, the composition of the invention rinses off rapidly while at the same time minimising the amount of unpleasant residues on the fibres and gives them, after rinsing, a natural and clean feel. The fibres treated with the composition of the invention also have good cosmetic properties, in particular in terms of softness, manageability, smoothness and feel. There are also well individualized and thus easier to disentangle.

A subject of the present invention is also a cosmetic process for treating, in particular for washing and/or for conditioning, keratin fibres, in particular human keratin fibres such as the hair, comprising the application to said keratin fibres of a solid composition as defined above, the solid composition being applied directly to said keratin fibres or after having been wetted beforehand with water.

The present invention also relates to the use of a solid composition as defined previously for washing and/or conditioning keratin fibres, in particular human keratin fibres such as the hair.

Other subjects, characteristics, aspects and advantages of the invention will become even more clearly apparent on reading the description and the example which follows.

In the text which follows, unless otherwise indicated, the limits of a range of values are included in that range, notably in the expressions “between” and “ranging from . . . to . . . ”.

Moreover, the expression “at least one” used in the present description is equivalent to the expression “one or more”.

Advantageously, the composition according to the invention comprises a water content of less than or equal to 15% by weight, preferably less than or equal to 12% by weight, better still less than or equal to 11% by weight, even better still less than or equal to 10% by weight, even better still less than or equal to 8% by weight, relative to the total weight of the composition. More particularly, the water content ranges from 0% to 15% by weight, preferably from 0% to 12% by weight, better still from 0% to 11% by weight, even better still from 0% to 10% by weight and even better still from 0% to 8% by weight, relative to the total weight of the composition.

In particular, the composition does not comprise water added during its preparation. The residual water possibly present can originate from the starting materials used during the preparation.

The solid composition according to the invention may be in the form of a powder, a paste, particles (for example spherical particles such as small balls or granules), a compressed tablet, a stick or a bar. Preferably, the composition according to the invention is in the form of a bar.

The term “powder” is intended to mean a composition in pulverulent form, which is preferably essentially free of dust (or fine particles). In other words, the particle size distribution of the particles is such that the weight ratio of particles less than or equal to 50 micrometres in size (fines content) and preferably less than or equal to 45 micrometres in size (fines content) is advantageously less than or equal to 5% by weight, preferably less than 3% by weight and more particularly less than 1% by weight, relative to the total weight of particles (particle size evaluated using a Retsch AS 200 Digit particle size analyser; oscillation height: 1.25 mm/screening time: 5 minutes).

The term “paste” is intended to mean a composition with a viscosity of greater than 5 poises and preferably greater than 10 poises, measured at 25° C. and at a shear rate of 1 s⁻¹; this viscosity may be determined using a cone-plate rheometer.

The term “particles” is intended to mean small fractionated objects formed from solid particles aggregated together, of variable shapes and sizes. They may be regular or irregular in shape. They may in particular have a spherical shape (such as granules, granular material, balls), a square shape, a rectangular shape, or an elongated shape such as rods. Spherical particles are most particularly preferred.

Advantageously, the size of the powders or of the particles is, in its largest dimension, between 45 μm and 5 mm, and more particularly between 50 μm and 2 mm, better still between 50 μm and 1 mm, even better still between 60 and 600 μm.

The term “bar” is intended to mean a finished product of variable shape and size, generally having a weight of between 10 and 200 g, of solid texture, composed of a mixture of ingredients which have been compressed/agglomerated or hot-cast. The mixture contains little air or is free of air.

When the solid composition according to the invention is in the form of a bar, said composition preferably has a penetration force, at 25° C., 1 atm, greater than or equal to 210 g, preferably greater than or equal to 500 g. The penetration force is determined by penetrometry, using a Stable Micro Systems TA.XT Plus connect texture analyser (Swantech) at 25° C. The penetrometry experiments are carried out with a metal rod fitted with a screwed probe of P/2N needle type, having a diameter of 2 mm at the top part, connected to the measuring head. The piston sinks into the sample at a constant speed of 1 mm/s over a height of 5 mm. The force exerted on the piston is recorded and the average value of the force is calculated.

The solid composition according to the invention can be in the form of a compressed solid composition, in particular compressed using a manual or mechanical press. Preferably, the hardness of the compressed solid composition is between 10 and 300 N, more preferentially between 15 and 200 N, and even better still between 15 and 100 N.

The density of the solid composition according to the present invention is preferably between 0.5 and 1.5, more preferentially between 0.9 and 1.3, and even better still between 1.0 and 1.2.

Anionic Surfactants of Sulfate Type

The solid composition according to the present invention comprises one or more anionic surfactants of sulfate type.

For the purposes of the present invention, the term “anionic surfactant of sulfate type” is intended to mean an anionic surfactant comprising one or more sulfate functions (—OSO₃H or —OSO₃—).

Such surfactants may advantageously be chosen from alkyl sulfates, alkyl ether sulfates, alkylamido sulfates, alkylamido ether sulfates, alkylaryl polyether sulfates, monoglyceride sulfates; and salts thereof and mixtures thereof; the alkyl groups of these compounds comprising in particular from 8 to 30 carbon atoms, preferably from 8 to 26, and more preferentially from 10 to 22 carbon atoms; the aryl group preferably denoting a phenyl or benzyl group; it being possible for these compounds to be polyoxyalkylenated, in particular polyoxyethylenated, and then preferably comprising from 1 to 50 ethylene oxide units, and more preferentially from 2 to 10 ethylene oxide units.

Preferably, the anionic surfactant(s) of sulfate type are chosen from:
alkyl sulfates, in particular C₈ to C₂₆, and preferably C₁₀ to C₂₂, alkyl sulfates;
alkyl ether sulfates, in particular C₈ to C₂₆, and preferably C₁₀ to C₂₂, alkyl ether sulfates, preferably comprising from 2 to 10 ethylene oxide units;
   in particular in the form of alkali metal, alkaline earth metal, ammonium or amino alcohol salts, and
   mixtures thereof.

When the anionic surfactant(s) of sulfate type are in salt form, said salt may be chosen from alkali metal salts, such as the sodium or potassium salt, ammonium salts, amine salts and in particular amino alcohol salts, and alkaline-earth metal salts, such as the magnesium salt, and mixtures thereof.

Mention may be made, as examples of amino alcohol salts, of mono-, di- and triethanolamine salts, mono-, di- or triisopropanolamine salts, 2-amino-2-methyl-1-propanol salts, 2-amino-2-methyl-1,3-propanediol salts and tris(hydroxymethyl)aminomethane salts.

The alkali metal or alkaline earth metal salts and in particular the sodium or magnesium salts are preferably used.

Preferably, the anionic surfactant(s) of sulfate type are chosen from sodium, triethanolamine, magnesium or ammonium (C₁₀-C₂₂)alkyl sulfates, sodium, ammonium or magnesium (C₁₀-C₂₂)alkyl ether sulfates, which are oxyethylenated, for example with 1 or 2.2 mol of ethylene oxide, and mixtures thereof.

Even better still, the anionic surfactant(s) of sulfate type are chosen from sodium, triethanolamine, magnesium or ammonium (C₁₀-C₂₂)alkyl sulfates.

Advantageously, the anionic surfactant(s) of sulfate type are chosen from sodium lauryl sulfate and sodium cocosulfate.

The total amount of the anionic surfactant(s) of sulfate type, present in the solid composition according to the invention, ranges from 10% to 35% by weight, preferably from 10% to 30% by weight, and more preferentially from 10% to 25% by weight, and even better still from 15% to 25% by weight, relative to the total weight of the composition.

Advantageously, the total amount of the anionic surfactant(s) of sulfate type chosen from (C₁₀-C₂₂)alkyl sulfates, present in the solid composition according to the invention, ranges from 10% to 35% by weight, preferably from 10% to 30% by weight, and more preferentially from 10% to 25% by weight, even better still from 15% to 25% by weight, relative to the total weight of the composition.

The presence of anionic surfactant(s) of sulfate type according to the amounts defined above in the solid composition of the present invention makes it possible to generate an abundant foam, presenting moreover interesting foaming qualities. The foam is indeed very stable, it holds a long time on the head, and it breaks less quickly than usual foam. In addition, the foam is more enveloping, thicker and more unctuous.

Anionic Surfactants of Sulfonate Type

The solid composition according to the present invention comprises one or more anionic surfactants of sulfonate type.

For the purposes of the present invention, the term “anionic surfactant of sulfonate type” is intended to mean an anionic surfactant comprising one or more sulfonic or sulfonate functions (—SO₃H or —SO₃⁻), which can optionally comprise one or more carboxylic or carboxylate functions (—COOH or —COO—) and which do not comprise sulfate functions.

Such surfactants may advantageously be chosen from alkyl sulfonates, alkylamide sulfonates, alkylaryl sulfonates, alpha-olefin sulfonates, paraffin sulfonates, alkylsulfosuccinates, alkyl ether sulfosuccinates, alkylamide sulfosuccinates, alkyl sulfoacetates, N-acyltaurates, acylisethionates, and also salts thereof and mixtures thereof; the alkyl groups of these compounds comprising in particular from 8 to 30 carbon atoms, preferably from 8 to 26 and more preferentially from 10 to 22 carbon atoms; the aryl group preferably denoting a phenyl or benzyl group; it being possible for these compounds to be polyoxyalkylenated, in particular polyoxyethylenated and then preferably comprising from 1 to 50 ethylene oxide units, and more preferentially from 2 to 10 ethylene oxide units.

Preferably, the anionic surfactant(s) of sulfonate type are chosen from N-acyltaurates, and in particular N-acyl N-methyl taurates, acylisethionates, and also salts thereof and mixtures thereof.

More preferentially, the anionic surfactant(s) of sulfonate type can be advantageously chosen from the compounds of formula (I) below:

R₁—COX—R₂—SO₃M (I),

in which:
R₁ represents a linear or branched, preferably linear, alkyl group comprising from 8 to 30 carbon atoms, preferably from 8 to 26 carbon atoms, and more preferentially from 10 to 22 carbon atoms;
X represents an oxygen atom or an —N(CH₃)— or —NH— group, preferably an oxygen atom;
R₂ represents a linear or branched alkyl group comprising from 1 to 4 carbon atoms; and
M denotes a hydrogen atom, an ammonium ion, an ion derived from an alkali metal or an alkaline-earth metal or an ion derived from an organic amine.

The anionic surfactant(s) of sulfonate type, and in particular those of formula (I) as defined above, can be used in salified or non-salified form.

Salts that may be used in particular include alkali metal salts such as sodium or potassium salts, ammonium salts, amine salts, amino alcohol salts or alkaline-earth metal salts, for example magnesium salts.

Amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

The alkali metal or alkaline earth metal salts and in particular the sodium or magnesium salts are preferably used.

Preferably, the anionic surfactant(s) of sulfonate type are chosen from acylisethionates, and more preferentially from (C₈-C₃₀)acylisethionates used in the form of salts, and even better still in the form of alkali metal salts or alkaline earth metal salts, and in particular sodium or magnesium salts.

As examples of (C₈-C₃₀)acylisethionates that are particularly preferred, mention may be made in particular of cocoyl isethionates and lauroyl methyl isethionates, in particular in the form of sodium salts.

Advantageously, the total amount of the anionic surfactant(s) of sulfonate type, present in the solid composition according to the invention, ranges from 5% to 45% by weight, preferably from 10% to 40% by weight, more preferentially from 20% to 35% by weight, even better still from 25% to 35% by weight, relative to the total weight of the composition.

In one preferred variant of the invention, the anionic surfactant(s) of sulfonate type are chosen from (C₈-C₃₀)acyl isethionates and mixtures thereof, and the total amount of the (C₈-C₃₀)acyl isethionate(s), present in the solid composition according to the invention, preferably ranges from 5% to 45% by weight, more preferentially from 10% to 40% by weight, even more preferentially from 20% to 35% by weight, even better still from 25% to 35% by weight, relative to the total weight of the composition.

According to one particular embodiment, the solid composition according to the invention is free of anionic surfactant of carboxylate type.

For the purposes of the present invention, the term “anionic surfactant of carboxylate type” is intended to mean an anionic surfactant comprising one or more carboxylic or carboxylate functions (—COOH or —COO—), and not comprising any sulfonic or sulfonate functions and not comprising any sulfate function.

For the purposes of the present invention, the term “free of” refers to a composition which does not contain (0%) these anionic surfactants of carboxylate type or which contains less than 0.1% by weight of such surfactants, relative to the total weight of the composition.

The total amount of the anionic surfactant(s), that is to say in particular the total amount of anionic surfactants of sulfate type a) and of anionic surfactants of sulfonate type b), present in the solid composition according to the invention, is preferably greater than or equal to 20% by weight; more preferentially, this amount ranges from 30% to 70% by weight, and even better still from 40% to 60% by weight, relative to the total weight of the composition.

Cationic Polymers

The solid composition according to the present invention also comprises one or more cationic polymers.

The cationic polymer(s) can be chosen from associative cationic polymers and non-associative cationic polymers, and mixtures thereof.

For the purposes of the present invention, the term “cationic polymer” is intended to mean any polymer comprising cationic groups and/or groups which can be ionized to cationic groups. Preferably, the cationic polymer(s) are hydrophilic or amphiphilic.

The cationic polymers are preferably not silicone polymers (not comprising any Si—O unit).

The preferred cationic polymers are chosen from those that contain units including primary, secondary, tertiary and/or quaternary amine groups that may either form part of the main polymer chain or may be borne by a side substituent directly connected thereto.

Preferably, the cationic polymers according to the invention do not comprise any anionic group nor any group that can be ionized to anionic groups.

The cationic polymers that may be used preferably have a weight-average molar mass (Mw) of between 500 and 5×10⁶ approximately and preferably between 10³ and 3×10⁶ approximately.

Advantageously, the cationic polymer(s) are non-associative and are chosen from:
(1) homopolymers or copolymers derived from acrylic or methacrylic esters or amides and including at least one of the units having the following formulae:

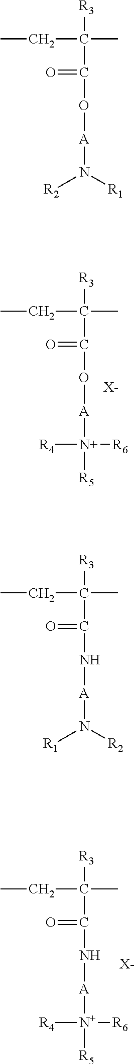

in which:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical, and preferably an alkyl group containing from 1 to 6 carbon atoms;
R₁ and R₂, which may be identical or different, represent a hydrogen atom or an alkyl group containing from 1 to 6 carbon atoms, and preferably methyl or ethyl; and
X denotes an anion derived from a mineral or organic acid, such as a methosulfate anion or a halide such as chloride or bromide.

The copolymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Among these copolymers of family (1), mention may be made of:
copolymers of acrylamide and of dimethylaminoethyl methacrylate quaternized with dimethyl sulfate or with a dimethyl halide, such as the product sold under the name Hercofloc by Hercules,
copolymers of acrylamide and of methacryloyloxyethyltrimethylammonium chloride, such as those sold under the name Bina Quat P 100 by Ciba Geigy,
the copolymer of acrylamide and methacryloyloxyethyltrimethylammonium methosulfate, such as the product sold under the name Reten by Hercules;
quaternized or non-quaternized vinylpyrrolidone/dialkylaminoalkyl acrylate or methacrylate copolymers, such as the products sold under the name Gafquat by ISP, for instance Gafquat 734 or Gafquat 755, or alternatively the products known as Copolymer 845, 958 and 937. These polymers are described in detail in French patents 2 077 143 and 2 393 573;
dimethylaminoethyl methacrylate/vinylcaprolactam/vinylpyrrolidone terpolymers, such as the product sold under the name Gaffix VC 713 by ISP;
vinylpyrrolidone/methacrylamidopropyldimethylamine copolymers, such as those sold under the name Styleze CC 10 by ISP;
quaternized vinylpyrrolidone/dimethylaminopropylmethacrylamide copolymers such as the product sold under the name Gafquat HS 100 by ISP;
the preferably crosslinked polymers of methacryloyloxy(C₁-C₄)alkyltri(C₁-C₄)alkylammonium salts, such as the polymers obtained by homopolymerization of dimethylaminoethylmethacrylate quaternized with methyl chloride or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. Use may be made more particularly of a crosslinked acrylamide/methacryloyloxyethyltrimethylammonium chloride copolymer (20/80 by weight) in the form of a dispersion comprising 50% by weight of said copolymer in mineral oil. This dispersion is sold under the name Salcare® SC 92 by Ciba. Use may also be made of a crosslinked methacryloyloxyethyltrimethylammonium chloride homopolymer comprising approximately 50% by weight of the homopolymer in mineral oil or in a liquid ester. These dispersions are sold under the names Salcare® SC 95 and Salcare® SC 96 by Ciba;
(2) cationic polysaccharides, notably cationic celluloses and galactomannan gums. Among the cationic polysaccharides, mention may be made more particularly of cellulose ether derivatives including quaternary ammonium groups, cationic cellulose copolymers or cellulose derivatives grafted with a water-soluble quaternary ammonium monomer and cationic galactomannan gums.

The cellulose ether derivatives including quaternary ammonium groups are notably described in FR 1 492 597, and mention may be made of the polymers sold under the name Ucare Polymer JR (JR 400 LT, JR 125 and JR 30M) or LR (LR 400 and LR 30M) by Amerchol. These polymers are also defined in the CTFA dictionary as quaternary ammoniums of hydroxyethylcellulose that have reacted with an epoxide substituted with a trimethylammonium group. By way of example, mention may in particular be made of hydroxyethylcellulose having a trimethylammonium group, preferably polyquaternium-10 (INCI name).

Cationic cellulose copolymers or cellulose derivatives grafted with a water-soluble quaternary ammonium monomer are described notably in patent U.S. Pat. No. 4,131,576, and mention may be made of hydroxyalkyl celluloses, for instance hydroxymethyl, hydroxyethyl or hydroxypropyl celluloses notably grafted with a methacryloylethyltrimethylammonium, methacrylamidopropyltrimethylammonium or dimethyldiallylammonium salt. The commercial products corresponding to this definition are more particularly the products sold under the names Celquat L 200 and Celquat H 100 by National Starch.

The cationic galactomannan gums are described more particularly in patents U.S. Pat. Nos. 3,589,578 and 4,031,307, and mention may be made of guar gums comprising cationic trialkylammonium groups. Use is made, for example, of guar gums modified with a 2,3-epoxypropyltrimethylammonium salt (for example, a chloride). Mention may in particular be made of guar hydroxypropyltrimethylammonium chloride. Such products are notably sold under the names Jaguar C13 S, Jaguar C 15, Jaguar C 17 and Jaguar C162 by Rhodia;
(3) polymers constituted of piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers;
(4) water-soluble polyaminoamides prepared in particular by polycondensation of an acidic compound with a polyamine. These polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they include one or more tertiary amine functions, they can be quaternized;
(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical includes from 1 to 4 carbon atoms and preferably denotes methyl, ethyl or propyl. Among these derivatives, mention may be made more particularly of the adipic acid/dimethylaminohydroxypropyl/diethylenetriamine polymers sold under the name Cartaretine F, F4 or F8 by Sandoz;
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms; the mole ratio between the polyalkylene polyamine and the dicarboxylic acid preferably being between 0.8:1 and 1.4:1; the resulting polyaminoamide being reacted with epichlorohydrin in a mole ratio of epichlorohydrin relative to the secondary amine group of the polyaminoamide preferably of between 0.5:1 and 1.8:1. Polymers of this type are sold in particular under the name Hercosett 57 by Hercules Inc. or else under the name PD 170 or Delsette 101 by Hercules in the case of the adipic acid/epoxypropyl/diethylenetriamine copolymer;
(7) cyclopolymers of alkyldiallylamine or of dialkyldiallylammonium, such as the homopolymers or copolymers containing, as main constituent of the chain, units corresponding to formula (VI) or (VII):

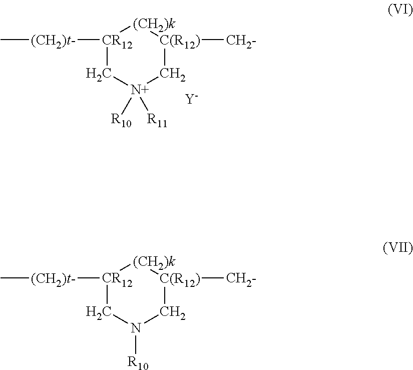

in which:
k and t are equal to 0 or 1, the sum k+t being equal to 1;
R₁₂ denotes a hydrogen atom or a methyl radical;
R₁₀ and R₁₁, independently of each other, denote an alkyl group containing from 1 to 6 carbon atoms, a hydroxyalkyl group in which the alkyl group contains 1 to 5 carbon atoms, a C₁-C₄ amidoalkyl group; or alternatively R₁₀ and R₁₁ may denote, together with the nitrogen atom to which they are attached, heterocyclic groups such as piperidinyl or morpholinyl; R₁₀ and R₁₁, independently of each other, preferably denote an alkyl group containing from 1 to 4 carbon atoms; and
Y⁻ is an anion such as bromide, chloride, acetate, borate, citrate, tartrate, bisulfate, bisulfite, sulfate or phosphate.

Mention may be made more particularly of the dimethyldiallylammonium salt (for example chloride) homopolymer, for example sold under the name Merquat 100 by Nalco (and homologs thereof of low weight-average molar masses) and the copolymers of diallyldimethylammonium salts (for example chloride) and of acrylamide, sold especially under the names Merquat 550 and Merquat 7SPR;
(8) diquaternary ammonium polymers comprising repeating units of formula (VI):

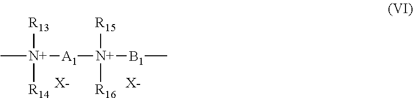

in which:
R₁₃, R₁₄, R₁₅ and R₁₆, which may be identical or different, represent aliphatic, alicyclic or arylaliphatic radicals comprising from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second heteroatom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a saturated or unsaturated and linear or branched alkylene or hydroxyalkylene radical, B₁ can also denote a (CH₂)ₙ—CO-D-OC—(CH₂)ₙ— group in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂—CH(CH₃)—O]_{y}—CH₂—CH(CH₃)—, where x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, in which Y denotes a linear or branched hydrocarbon-based radical, or alternatively the divalent radical —CH₂—CH₂—S—S—CH₂—CH₂—,
d) a ureylene group of formula: —NH—CO—NH—.

Preferably, X⁻ is an anion, such as chloride or bromide. These polymers have a number-average molar mass (Mn) generally of between 1000 and 100 000.

Mention may be made more particularly of polymers constituted of repeating units corresponding to the formula:

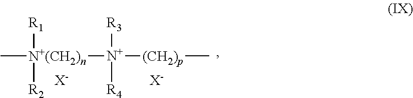

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid.

A compound of formula (IX) that is particularly preferred is the one for which R₁, R₂, R₃ and R₄ represent a methyl radical, n=3, p=6 and X=Cl, which is known as Hexadimethrine chloride according to the INCI (CTFA) nomenclature;
(9) polyquaternary ammonium polymers comprising units of formula (VIII):

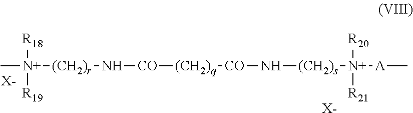

in which:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom;
r and s, which may be identical or different, are integers between 1 and 6;
q is equal to 0 or to an integer between 1 and 34;
X⁻ denotes an anion, such as a halide, and
A denotes a radical of a dihalide or preferably represents —CH₂—CH₂—O—CH₂—CH₂—.

Examples that may be mentioned include the products Mirapol® A 15, Mirapol® AD1, Mirapol® AZ1 and Mirapol® 175 sold by Miranol;
(10) quaternary polymers of vinylpyrrolidone and of vinylimidazole, for instance the products sold under the names Luviquat® FC 905, FC 550 and FC 370 by BASF;
(11) polyamines such as Polyquart® H sold by Cognis, referred to under the name Polyethylene glycol (15) tallow polyamine in the CTFA dictionary; and
(12) polymers including in their structure:
   (a) one or more units corresponding to formula (A) below:

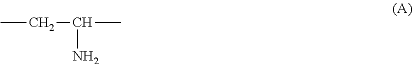

(b) optionally one or more units corresponding to formula (B) below:

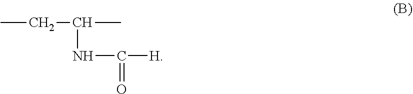

In other words, these polymers may be notably chosen from homopolymers or copolymers including one or more units derived from vinylamine and optionally one or more units derived from vinylformamide.

Preferably, these cationic polymers are chosen from polymers including, in their structure, from 5 mol % to 100 mol % of units corresponding to formula (A) and from 0 to 95 mol % of units corresponding to formula (B), preferentially from 10 mol % to 100 mol % of units corresponding to formula (A) and from 0 to 90 mol % of units corresponding to formula (B).

These polymers may be obtained, for example, by partial hydrolysis of polyvinylformamide. This hydrolysis may take place in acidic or basic medium.

The weight-average molecular weight of said polymer, measured by light scattering, may range from 1000 to 3 000 000 g/mol, preferably from 10 000 to 1 000 000 and more particularly from 100 000 to 500 000 g/mol.

The cationic charge density of these polymers may range from 2 meq/g to 20 meq/g, preferably from 2.5 to 15 meq/g and more particularly from 3.5 to 10 meq/g.

The polymers including units of formula (A) and optionally units of formula (B) are notably sold under the name Lupamin by BASF, for instance, in a non-limiting manner, the products provided under the names Lupamin 9095, Lupamin 5095, Lupamin 1095, Lupamin 9030 (or Luviquat 9030) and Lupamin 9010.

Advantageously, the cationic polymers are associative.

For the purposes of the present invention, the term “associative polymers” means polymers that are capable, in an aqueous medium, of reversibly combining with each other or with other molecules.

Their chemical structure more particularly comprises at least one hydrophilic group and at least one hydrophobic group.

The term “hydrophobic group” means a radical or polymer with a saturated or unsaturated, linear or branched hydrocarbon-based chain, comprising at least 8 carbon atoms, preferably from 8 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative cationic polymers that may be used, alone or as a mixture, mention may be made of:
(A) cationic associative polyurethanes, which may be represented by the general formula (Ia) below:

R—X—(P)n-[L-(Y)m]r-L′-(P′)p—X′—R′ (Ia),

in which:
R and R′, which may be identical or different, represent a hydrophobic group or a hydrogen atom;
X and X′, which may be identical or different, represent a group comprising an amine function optionally bearing a hydrophobic group, or alternatively a group L″;
L, L′ and L″, which may be identical or different, represent a group derived from a diisocyanate;
P and P′, which may be identical or different, represent a group comprising an amine function optionally bearing a hydrophobic group;
Y represents a hydrophilic group;
r is an integer between 1 and 100 inclusive, preferably between 1 and 50 inclusive and in particular between 1 and 25 inclusive;
n, m and p are each, independently of one another, between 0 and 1000 inclusive;
the molecule containing at least one protonated or quaternized amine function and at least one hydrophobic group.

Preferably, the only hydrophobic groups are the groups R and R′ at the chain ends.

One preferred family of cationic associative polyurethanes is the one corresponding to formula (Ia) described above, in which:
R and R′ both independently represent a hydrophobic group,
X and X′ each represent a group L″,
n and p are integers that are between 1 and 1000 inclusive, and L, L′, L″, P, P′, Y and m have the meaning indicated above.

Another preferred family of cationic associative polyurethanes is the one corresponding to formula (Ia) above in which:
n=p=0 (the polymers do not include any units derived from a monomer containing an amine function, incorporated into the polymer during the polycondensation),
the protonated amine functions result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide or a sulfate.

Yet another preferred family of cationic associative polyurethanes is the one corresponding to formula (Ia) above in which:
R and R′ both independently represent a hydrophobic group,
X and X′ both independently represent a group comprising a quaternary amine,
n=p=0, and
L, L′, Y and m have the meaning given above.

The number-average molecular weight (Mn) of the cationic associative polyurethanes is preferably between 400 and 500 000 inclusive, in particular between 1000 and 400 000 inclusive and ideally between 1000 and 300 000 inclusive.

The term “hydrophobic group” is intended to mean a radical or polymer containing a saturated or unsaturated, linear or branched hydrocarbon-based chain, which may contain one or more heteroatoms such as P, O, N or S, or a radical containing a perfluoro or silicone chain. When the hydrophobic group denotes a hydrocarbon-based radical, it includes at least 8 carbon atoms, preferably from 8 to 30 carbon atoms, in particular from 12 to 30 carbon atoms and more preferentially from 18 to 30 carbon atoms.

Preferentially, the hydrocarbon-based group is derived from a monofunctional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group comprising a tertiary or quaternary amine, X and/or X′ may represent one of the following formulae:

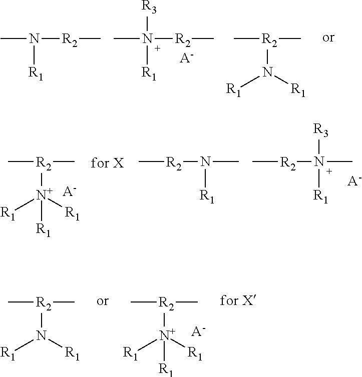

in which:
R₂ represents a linear or branched alkylene radical containing from 1 to 20 carbon atoms, optionally including a saturated or unsaturated ring, or an arylene radical, one or more of the carbon atoms possibly being replaced with a heteroatom chosen from N, S, O and P;
R₁ and R₃, which may be identical or different, denote a linear or branched C₁-C₃₀ alkyl or alkenyl radical or an aryl radical, at least one of the carbon atoms possibly being replaced with a heteroatom chosen from N, S, O and P;
A⁻ is a physiologically acceptable anionic counterion such as a halide, for instance a chloride or bromide, or a mesylate.

The groups L, L′ and L″ represent a group of formula:

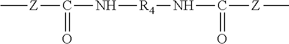

in which:
Z represents —O—, —S— or —NH—; and
R₄ represents a linear or branched alkylene radical containing from 1 to 20 carbon atoms, optionally including a saturated or unsaturated ring, or an arylene radical, one or more of the carbon atoms possibly being replaced with a heteroatom chosen from N, S, O and P.

The groups P and P′ comprising an amine function may represent at least one of the following formulae:

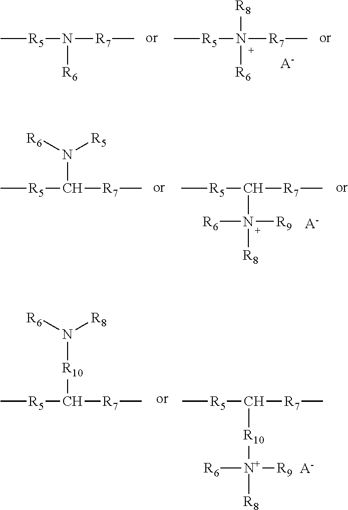

in which:
R₅ and R₇ have the same meanings as R₂ defined previously;
R₆, R₈ and R₉ have the same meanings as R₁ and R₃ defined previously;
R₁₀ represents a linear or branched, optionally unsaturated alkylene group possibly containing one or more heteroatoms chosen from N, O, S and P;
and A⁻ is a physiologically acceptable anionic counterion such as a halide, for instance chloride or bromide, or mesylate.

As regards the meaning of Y, the term “hydrophilic group” is intended to mean a polymeric or non-polymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. The hydrophilic compound is preferentially a polyether and in particular a poly(ethylene oxide) or poly(propylene oxide).

The associative cationic polyurethanes of formula (Ia) are formed from diisocyanates and from various compounds bearing functions containing a labile hydrogen. The functions containing labile hydrogen may be alcohol, primary or secondary amine or thiol functions, giving, after reaction with the diisocyanate functions, polyurethanes, polyureas and polythioureas, respectively. In the present invention, the term “polyurethanes” encompasses these three types of polymer, namely polyurethanes per se, polyureas and polythioureas, and also copolymers thereof.

A first type of compound involved in the preparation of the polyurethane of formula (Ia) is a compound comprising at least one unit bearing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may include more than one unit containing an amine function. In this case, it is a polymer bearing a repetition of the unit containing an amine function.

Compounds of this type may be represented by one of the following formulae:

HZ—(P)ₙ—ZH, or HZ—(P′)ₚ—ZH, in which Z, P, P′, n and p are as defined above.

Examples that may be mentioned include N-methyldiethanolamine, N-tert-butyldiethanolamine and N-sulfoethyldiethanolamine.

The second compound included in the preparation of the polyurethane of formula (Ia) is a diisocyanate corresponding to the formula:

O═C═N—R₄—N═C═O in which R₄ is as defined above.

By way of example, mention may be made of methylenediphenyl diisocyanate, methylenecyclohexane diisocyanate, isophorone diisocyanate, tolylene diisocyanate, naphthalene diisocyanate, butane diisocyanate and hexane diisocyanate.

A third compound involved in the preparation of the polyurethane of formula (Ia) is a hydrophobic compound intended to form the terminal hydrophobic groups of the polymer of formula (Ia).

This compound is formed from a hydrophobic group and a function containing a labile hydrogen, for example a hydroxyl, primary or secondary amine, or thiol function.

By way of example, this compound may be a fatty alcohol such as stearyl alcohol, dodecyl alcohol or decyl alcohol. When this compound includes a polymeric chain, it may be, for example, α-hydroxylated hydrogenated polybutadiene.

The hydrophobic group of the polyurethane of formula (Ia) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide, a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing labile hydrogen are alcohol, primary or secondary amine or thiol functions. This compound may be a polymer terminated at the chain ends with one of these functions containing labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and in particular a poly(ethylene oxide) or poly(propylene oxide).

The hydrophilic group termed Y in formula (Ia) is optional. Specifically, the units containing a quaternary or protonated amine function may suffice to provide the solubility or water-dispersibility required for this type of polymer in an aqueous solution.

Although the presence of a hydrophilic group Y is optional, cationic associative polyurethanes comprising such a group are, however, preferred.
(B) quaternized cellulose derivatives, and in particular quaternized celluloses modified with groups including at least one fatty chain, such as linear or branched alkyl groups, linear or branched arylalkyl groups, or linear or branched alkylaryl groups, preferably linear or branched alkyl groups, these groups including at least 8 carbon atoms, notably from 8 to 30 carbon atoms, better still from 10 to 24, or even from 10 to 14, carbon atoms; or mixtures thereof.

Preferably, mention may be made of quaternized hydroxyethylcelluloses modified with groups comprising at least one fatty chain, such as linear or branched alkyl groups, linear or branched arylalkyl groups, or linear or branched alkylaryl groups, preferably linear or branched alkyl groups, these groups comprising at least 8 carbon atoms, in particular from 8 to 30 carbon atoms, better still from 10 to 24, or even from 10 to 14, carbon atoms; or mixtures thereof.

Preferentially, mention may be made of the hydroxyethylcelluloses of formula (Ib):

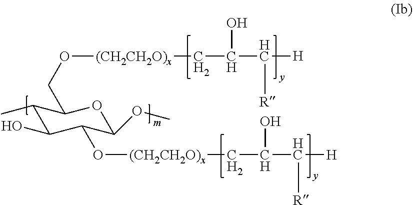

in which:
R represents an ammonium group RaRbRcN⁺—, Q⁻ in which Ra, Rb and Rc, which may be identical or different, represent a hydrogen atom or a linear or branched C₁-C₃₀ alkyl, and Q⁻ represents an anionic counterion such as a halide, for instance a chloride or bromide; preferably an alkyl;
R′ represents an ammonium group R′aR′bR′cN⁺—, Q′⁻ in which R′a, R′b and R′c, which may be identical or different, represent a hydrogen atom or a linear or branched C₁-C₃₀ alkyl, and Q′⁻ represents an anionic counterion such as a halide, for instance a chloride or bromide; preferably an alkyl;
it being understood that at least one of the radicals Ra, Rb, Rc, R′a, R′b and R′c represents a linear or branched C₈-C₃₀ alkyl;
n, x and y, which may be identical or different, represent an integer between 1 and 10 000.

Preferably, in formula (Ib), at least one of the radicals Ra, Rb, Rc, R′a, R′b or R′c represents a linear or branched C₈ to C₃₀, better still C₁₀-C₂₄ or even C₁₀-C₁₄ alkyl; mention may be made in particular of the dodecyl radical (C₁₂). Preferably, the other radical(s) represent a linear or branched C₁-C₄ alkyl, notably methyl.

Preferably, in formula (Ib), only one of the radicals Ra, Rb, Rc, R′a, R′b or R′c represents a linear or branched C₈-C₃₀, better still C₁₀-C₂₄ or even C₁₀-C₁₄ alkyl; mention may be made in particular of the dodecyl radical (C₁₂). Preferably, the other radicals represent a linear or branched C₁-C₄ alkyl, notably methyl.

Even better still, R may be a group chosen from —N⁺(CH₃)₃, Q′⁻ and —N⁺(C₁₂H₂₅)(CH₃)₂, Q′⁻, preferably an —N⁺(CH₃)₃, Q′⁻ group.

Even better still, R′ may be a group —N⁺(C₁₂H₂₅)(CH₃)₂, Q′⁻.

The aryl radicals preferably denote phenyl, benzyl, naphthyl or anthryl groups.

Mention may notably be made of the polymers having the following INCI names:
Polyquaternium-24, such as the product Quatrisoft LM 200®, sold by Amerchol/Dow Chemical;
PG-Hydroxyethylcellulose Cocodimonium Chloride, such as the product Crodacel QM®;
PG-Hydroxyethylcellulose Lauryldimonium Chloride (C₁₂ alkyl), such as the product Crodacel QL®; and
PG-Hydroxyethylcellulose Stearyldimonium Chloride (C₁₈ alkyl), such as the product Crodacel QS®, sold by Croda.

Mention may also be made of the hydroxyethylcelluloses of formula (Ib) in which R represents a trimethylammonium halide and R′ represents a dimethyldodecylammonium halide, preferentially R represents trimethylammonium chloride (CH₃)₃N⁺—, Cl⁻ and R′ represents dimethyldodecylammonium chloride (CH₃)₂(C₂H₂₅)N⁺—, Cl⁻. This type of polymer is known under the INCI name Polyquaternium-67; as commercial products, mention may be made of the Softcat Polymer SL® polymers, such as SL-100, SL-60, SL-30 and SL-5, from Amerchol/Dow Chemical.

More particularly, the polymers of formula (Ib) are, for example, those whose viscosity is between 2000 and 3000 cPs inclusive, preferentially between 2700 and 2800 cPs. Typically, Softcat Polymer SL-5 has a viscosity of 2500 cPs, Softcat Polymer SL-30 has a viscosity of 2700 cPs, Softcat Polymer SL-60 has a viscosity of 2700 cPs and Softcat Polymer SL-100 has a viscosity of 2800 cPs. Use may also be made of Softcat Polymer SX-1300X with a viscosity of between 1000 and 2000 cPs.
(C) cationic polyvinyllactams, notably those comprising:
a) at least one monomer of vinyllactam or alkylvinyllactam type;
b) at least one monomer of structure (Ic) or (IIc) below:

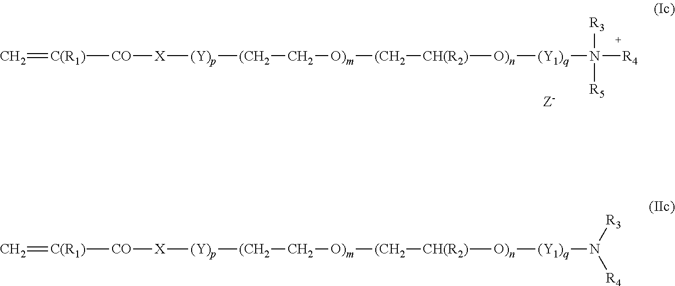

in which:
X denotes an oxygen atom or a radical NR₆,
R₁ and R₆ denote, independently of each other, a hydrogen atom or a linear or branched C₁-C₅ alkyl radical,
R₂ denotes a linear or branched C₁-C₄ alkyl radical,
R₃, R₄ and R₅ denote, independently of each other, a hydrogen atom, a linear or branched C₁-C₃₀ alkyl radical or a radical of formula (IIIc):

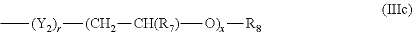

Y, Y₁ and Y₂ denote, independently of each other, a linear or branched C₂-C₁₆ alkylene radical,
R₇ denotes a hydrogen atom or a linear or branched C₁-C₄ alkyl radical or a linear or branched C₁-C₄ hydroxyalkyl radical,
R₈ denotes a hydrogen atom or a linear or branched C₁-C₃₀ alkyl radical,
p, q and r denote, independently of one another, 0 or 1,
m and n denote, independently of one another, an integer ranging from 0 to 100 inclusive,
x denotes an integer ranging from 1 to 100 inclusive,
Z denotes an anionic counterion of an organic or mineral acid, such as a halide, for instance chloride or bromide, or mesylate;
with the proviso that:
at least one of the substituents R₃, R₄, R₅ or R₈ denotes a linear or branched C₈-C₃₀ alkyl radical,
if m and/or n is other than zero, then q is equal to 1,
if m=n=0, then p or q is equal to 0.

Said cationic poly(vinyllactam) polymers may be crosslinked or noncrosslinked and may also be block polymers.

Preferably, the counterion Z⁻ of the monomers of formula (Ic) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferentially, the monomer b) is a monomer of formula (Ic) for which, preferentially, m and n are equal to 0.

The vinyllactam or alkylvinyllactam monomer is preferably a compound of structure (IVc):

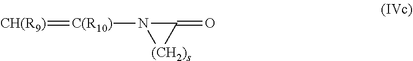

in which:
s denotes an integer ranging from 3 to 6,
R₉ denotes a hydrogen atom or a linear or branched C₁-C₅ alkyl radical,
R₁₀ denotes a hydrogen atom or a linear or branched C₁-C₅ alkyl radical,

with the proviso that at least one of the radicals R₉ and R₁₀ denotes a hydrogen atom.

Even more preferentially, the monomer (IVc) is vinylpyrrolidone.

The cationic poly(vinyllactam) polymers may also contain one or more additional monomers, preferably cationic or non-ionic monomers.

As compounds that are particularly preferred, mention may be made of the following terpolymers comprising at least:
a) one monomer of formula (IVc),
b) one monomer of formula (Ic) in which p=1, m=n=q=0, R₃ and R₄ denote, independently of each other, a hydrogen atom or a C₁-C₅ alkyl radical and R₅ denotes a linear or branched C₈-C₂₄ alkyl radical, and
c) one monomer of formula (IIc) in which p=1, m=n=q=0, R₃ and R₄ denote, independently of each other, a hydrogen atom or a linear or branched C₁-C₅ alkyl radical.

Even more preferentially, terpolymers comprising, by weight, 40% to 95% of monomer (a), 0.1% to 55% of monomer (c) and 0.25% to 50% of monomer (b) will be used. Such polymers are in particular described in patent application WO-00/68282.

As cationic poly(vinyllactam) polymers according to the invention, the following are in particular used:
vinylpyrrolidone/dimethylaminopropylmethacrylamide/dodecyldimethylmethacrylamidopropylammonium tosylate terpolymers,
vinylpyrrolidone/dimethylaminopropylmethacrylamide/cocoyldimethylmethacrylamidopropylammonium tosylate terpolymers,
vinylpyrrolidone/dimethylaminopropylmethacrylamide/lauryldimethylmethacrylamidopropylammonium tosylate or chloride terpolymers.

The vinylpyrrolidone/dimethylaminopropylmethacrylamide/lauryldimethylmethylacrylamidopropylammonium chloride terpolymer is in particular sold by ISP under the names Styleze W10® and Styleze W20L® (INCI name: Polyquaternium-55).

The weight-average molecular weight (Mw) of the cationic poly(vinyllactam) polymers is preferably between 500 and 20 000 000, more particularly between 200 000 and 2 000 000 and preferentially between 400 000 and 800 000.
(D) the cationic polymers obtained by polymerization of a monomer mixture comprising one or more vinyl monomers substituted with one or more amino groups, one or more hydrophobic non-ionic vinyl monomers, and one or more associative vinyl monomers, as described in patent application WO 2004/024779.

Among these copolymers, mention may be made more particularly of the products of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.

Such a polymer is, for example, the compound sold by Lubrizol under the name Carbopol Aqua CC® and which corresponds to the INCI name Polyacrylate-1 Crosspolymer.

Preferably, the cationic polymer(s) are chosen from cationic polysaccharides, quaternized (poly)hydroxyethylcelluloses modified with groups comprising at least one fatty chain, such as linear or branched alkyl groups, linear or branched arylalkyl groups, or linear or branched alkylaryl groups, preferably linear or branched alkyl groups, these groups comprising at least 10 carbon atoms, in particular from 10 to 30 carbon atoms, better still from 10 to 24 carbon atoms, and mixtures thereof.

More preferentially, the cationic polymers are chosen from non-associative celluloses, cationic galactomannan gums, quaternized (poly)hydroxyethylcelluloses modified with groups comprising at least one alkyl group containing at least 10 carbon atoms, preferentially ranging from 10 to 22 carbon atoms, and more preferentially ranging from 12 to 16 carbon atoms, and mixtures thereof, even more preferentially from cellulose ether derivatives comprising quaternary ammonium groups, cationic guar gums, quaternized (poly)hydroxyethylcelluloses modified with groups comprising at least one alkyl group containing at least 10 carbon atoms, preferentially ranging from 10 to 22 carbon atoms, and more preferentially ranging from 12 to 16 carbon atoms, and mixtures thereof.

In one preferred embodiment, the cationic polymer(s) are chosen from cationic galactomannan gums, preferably from cationic guar gums.

Advantageously, the total amount of the cationic polymer(s) ranges from 0.05% to 20% by weight, preferably ranges from 0.1% to 10% by weight, more preferentially from 0.5% to 8% by weight, even better still from 1% to 5% by weight, even better still from 1% to 3% by weight, or even from 2% to 3% by weight, relative to the total weight of the composition.

In one particular embodiment, advantageously, the total amount of the cationic polymer(s) chosen from cationic guar gums ranges from 0.05% to 20% by weight, preferably ranges from 0.1% to 10% by weight, more preferentially from 0.5% to 8% by weight, even better still from 1% to 5% by weight, even better still from 1% to 3% by weight, or even from 2% to 3% by weight, relative to the total weight of the composition.

(Poly)glycerol Esters

The solid composition according to the present invention also comprises one or more (poly)glycerol esters.

Advantageously, the (poly)glycerol ester(s) are chosen from:
monoesters or polyesters of linear or branched C₈ to C₄₀ acids, which are monoglycerolated or polyglycerolated, comprising from 1 to 50 mol of glycerol, preferably from 1 to 20 or even from 2 to 10 mol of glycerol; in particular monoesters or diesters of linear or branched C₈ to C₃₂, better still C₁₀ to C₂₈ or even C₁₀ to C₂₄ acids, comprising from 1 to 50 mol of glycerol, preferably from 1 to 20 or even from 2 to 10 mol of glycerol,
   and more preferentially from monoesters or diesters of linear or branched C₈ to C₄₀, better still C₈ to C₃₂, even better still C₁₀ to C₂₈ or even C₁₀ to C₂₄ acids, which are monoglycerolated or polyglycerolated, comprising from 1 to 50 mol of glycerol, preferably from 1 to 20 or even from 2 to 10 mol of glycerol;
triglycerides constituted of esters of fatty acids and glycerol, the fatty acids of which may be C₆ to C₂₄, preferably C₈-C₂₂, fatty acids;
mixtures thereof.

Among the mono- or diesters of C₈ to C₄₀ acids, mention may in particular be made of:
diesters of branched C₁₂ to C₃₂, better still C₁₄ to C₂₈, or even C₁₆ to C₂₄ acids, comprising from 1 to 50 mol of glycerol, preferably from 1 to 20 or even from 2 to 10 mol of glycerol; preferentially diesters of branched C₁₆ to C₂₄ acids, comprising from 2 to 10 mol of glycerol, among which mention may be made of isostearic acid diester containing 3 mol of glycerol (INCI name: Polyglyceryl-3 diisostearate);
monoesters of linear or branched, preferably linear, C₈ to C₂₄, better still C₁₀ to C₂₀, or even C₁₀ to Cis acids, comprising from 1 to 50 mol of glycerol, preferably from 1 to 20 or even from 2 to 10 mol of glycerol; preferentially monoesters of linear C₁₀ to Cis acids, comprising from 2 to 10 mol of glycerol, among which mention may be made of lauric acid monoester containing 4 mol of glycerol (INCI name: Polyglyceryl-4 laurate);
and mixtures thereof.

As indicated above, the (poly)glycerol ester(s) may be chosen from triglycerides constituted of esters of fatty acids and glycerol, the fatty acids of which may be C₆ to C₂₄, preferably C₈-C₂₂, fatty acids.

These varied C₆ to C₂₄ chains may be linear or branched, and saturated or unsaturated.

These particular (poly)glycerol esters may in particular be chosen from heptanoic or octanoic triglycerides, plant oils such as sweet almond oil, argan oil, avocado oil, groundnut oil, camellia oil, safflower oil, beauty-leaf oil, rapeseed oil, coconut (copra) oil, coriander oil, marrow oil, wheatgerm oil, jojoba oil, linseed oil, macadamia oil, corn germ oil, hazelnut oil, walnut oil, vernonia oil, apricot kernel oil, olive oil, evening primrose oil, palm oil, passion flower oil, grapeseed oil, rose oil, castor oil, rye oil, sesame oil, rice bran oil, camelina oil, soybean oil, sunflower oil, pracaxi oil, babassu oil, mongongo oil, marula oil, arara oil, shea butter oil, Brazil nut oil, hydrogenated plant oils, such as for example hydrogenated palm oil, hydrogenated castor oil; or alternatively caprylic/capric acid triglycerides, for instance those sold by Stearineries Dubois or those sold under the names Miglyol 810®, 812® and 818® by Dynamit Nobel, and the refined plant perhydrosqualene sold under the name Fitoderm by Cognis; the plant squalene sold, for example, under the name Squalive by Biosynthis.

Particularly preferably, the triglycerides constituted of esters of fatty acids and glycerol, the fatty acids of which may be C₆ to C₂₄ fatty acids, are chosen from caprylic/capric acid triglycerides, hydrogenated or non-hydrogenated plant oils, and mixtures thereof.

According to one particularly preferred embodiment, the (poly)glycerol esters are chosen from linear monoesters of C₁₀-C₁₈ acids comprising from 2 to 10 mol of glycerol, such as the lauric acid monoester comprising 4 mol of glycerol, caprylic/capric acid triglycerides, hydrogenated or non-hydrogenated plant oils, and mixtures thereof.

Advantageously, the total amount of the (poly)glycerol ester(s) ranges from 0.1% to 60% by weight, preferably from 1% to 50% by weight, more preferentially from 3% to 45% by weight, even more preferentially from 5% to 40% by weight, better still from 5% to 30% by weight, even better still from 5% to 20% by weight, relative to the total weight of the composition.

Polyols

The solid composition according to the present invention may also comprise one or more polyols.

The polyol(s), when they are present, are preferably chosen from sorbitol, glycerol, propylene glycol, and mixtures thereof, more preferentially from glycerol, sorbitol and mixtures thereof, and even better they represent a mixture of glycerol and sorbitol.

Advantageously, the total amount of the polyol(s), when they are present, ranges from 0.1% to 20% by weight, preferably from 0.5% to 15% by weight, more preferentially from 1% to 8% by weight, even better still from 1% to 5% by weight, relative to the total weight of the composition.

Anti-Dandruff Agents

The solid composition according to the present invention may also comprise one or more anti-dandruff agents.

The anti-dandruff agent(s), when they are present, are preferably chosen from pyrithione salts, 1-hydroxy-2-pyridone derivatives and selenium (poly)sulfides, and also mixtures thereof; more preferentially from 1-hydroxy-2-pyridone derivatives, selenium (poly)sulfides, and mixtures thereof.

Pyrithione is the compound 1-hydroxy-2(1H)-pyridinethione or 2-pyridinethiol-1-oxide.

The pyrithione salts which can be used in the context of the invention are in particular the monovalent metal salts and the divalent metal salts, such as the sodium, calcium, magnesium, barium, strontium, zinc, cadmium, tin and zirconium salts. The divalent metal salts and more particularly the zinc salt (zinc pyrithione) are particularly preferred.

The 1-hydroxy-2-pyridone derivatives are preferably chosen from compounds of formula (A1) or salts thereof:

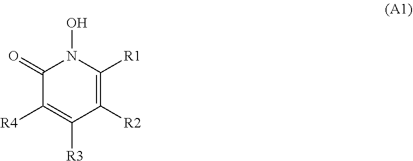

in which:
R1 denotes a hydrogen atom; a linear or branched alkyl group having from 1 to 17 carbon atoms; a cycloalkyl group having from 5 to 8 carbon atoms; a cycloalkyl-alkyl group, the cycloalkyl group having from 5 to 8 carbon atoms and the alkyl group having from 1 to 4 carbon atoms; an aryl or aralkyl group, the aryl group having from 6 to 30 carbon atoms and the alkyl group having from 1 to 4 carbon atoms; an aryl-alkenyl group, the aryl group having from 6 to 30 carbon atoms and the alkenyl group having from 2 to 4 carbon atoms; the cycloalkyl and aryl groups as defined above may be substituted by one or more alkyl groups having from 1 to 4 carbon atoms or else one or more alkoxy groups having from 1 to 4 carbon atoms;
R2 denotes a hydrogen atom; an alkyl group having from 1 to 4 carbon atoms; an alkenyl group having from 2 to 4 carbon atoms; a halogen atom or a benzyl group;
R3 denotes a hydrogen atom, an alkyl group having from 1 to 4 carbon atoms or a phenyl group; and
R4 denotes a hydrogen atom; an alkyl group having from 1 to 4 carbon atoms; an alkenyl group having from 2 to 4 carbon atoms; a methoxymethyl group; a halogen atom or a benzyl group.

Among these compounds, those which are particularly preferred are 1-hydroxy-4-methyl-6-(2,4,4-trimethylpentyl)-2-(1H)-pyridone and 6-cyclohexyl-1-hydroxy-4-methyl-2-(1H)-pyridone.

Among the salts which can be used, mention may be made of the salts of lower (C1-C4) alkanolamines, such as ethanolamine and diethanolamine, amine or alkylamine salts, and also salts with inorganic cations, for instance ammonium salts, alkali metal salts or alkaline-earth metal salts.

Preference will quite particularly be given to the monoethanolamine salt of 1-hydroxy-4-methyl-6-(2,4,4-trimethylpentyl)-2(1H)-pyridinone (or piroctone), more commonly referred to as piroctone olamine or octopirox.

Among the selenium (poly)sulfides, mention may be made of selenium disulfide and selenium polysulfides of formula SeₓS_{y} in which x and y are numbers such that x+y=8. The selenium disulfide is in the form of a powder, the particles of which generally have a particle size of less than 200 μm and preferably less than 25 μm.

The anti-dandruff agent(s), when they are present, are preferably chosen from piroctone olamine and selenium disulfide, and also a mixture thereof.

Advantageously, the total amount of the anti-dandruff agent(s), when they are present in the solid composition according to the invention, ranges from 0.01% to 1% by weight, preferably from 0.05% to 0.8% by weight and even better still from 0.1% to 0.5% by weight, relative to the total weight of the composition.

Additional Compounds

The solid composition according to the present invention can optionally also comprise one or more additional compounds other than the compounds defined above, preferably chosen from cationic surfactants, anionic or non-ionic polymers, and mixtures thereof, antioxidants, penetration agents, sequesters, fragrances, buffers, dispersants, conditioning agents such as, for example, modified or unmodified, volatile or non-volatile silicones, film-forming agents, ceramides, preserving agents, opacifiers, lubricants (or anti-caking agents), scrubs, colorants, nacreous agents and pigments, and mixtures thereof.

The composition according to the invention may be prepared by any means known to those skilled in the art, in particular by extrusion followed by optional cutting up of the extruded product in order to obtain a composition of the desired size (for example in the form of a bar), or else by compression optionally followed by cutting up, or else by moulding of the composition, in particular in moulds having the desired final shape or in other types of moulds, and followed by cutting up.

A subject of the present invention is also a cosmetic process for treating, and in particular a process for washing and/or for conditioning, keratin fibres, in particular human keratin fibres such as the hair, comprising the application to said keratin fibres of a solid composition as defined above, the solid composition being applied directly to said keratin fibres or after having been wetted beforehand with water.

The solid composition according to the invention may be applied to wet or dry keratin fibres, and preferably to wet keratin fibres.

The solid composition thus applied can optionally be rinsed off or left on, with an optional leave-on time that may range from 1 to 15 minutes, preferably from 2 to 10 minutes. Preferably, the composition according to the invention is rinsed off.

According to a first embodiment of the invention, the solid composition is applied directly to the keratin fibres, that is to say without being pre-wetted with water.

When, according to this first embodiment, the solid composition of the invention is applied directly (that is to say without prior wetting) to the dry keratin fibres, water may optionally be added to said fibres so as to subsequently rub/massage in order to solubilize/pre-emulsify said composition and to form an abundant and immediate foam. The foam thus obtained can subsequently be rinsed off after an optional leave-on time.

Conversely, the solid composition of the invention can also be applied directly (that is to say without prior wetting) to wet keratin fibres, followed by massaging/rubbing so as to obtain an immediate and abundant foam. The foam thus obtained can subsequently be rinsed off after an optional leave-on time.

According to another embodiment of the invention, the solid composition is pre-wetted in water before being applied to the keratin fibres. According to this embodiment, a small amount (preferably ranging from 1 to 3 g) of solid composition is advantageously solubilized with water, for example in the hand, so as to form an abundant and immediate foam. The foam thus obtained can then be applied to the wet or dry keratin fibres, before being optionally rinsed off with water after an optional leave-on time.

Finally, a subject of the present invention is the use of a solid composition as defined previously for washing and/or conditioning keratin fibres, in particular human keratin fibres such as the hair.

The following examples serve to illustrate the invention without, however, being limiting in nature.

### EXAMPLES

In the examples which follow, all the amounts are given, unless otherwise indicated, as percentage by weight of active material (g % AM), relative to the total weight of the composition.

Compositions

The compositions were prepared from the ingredients indicated in the tables below (g %):

TABLE 1

| Compositions | A | B | C |
|---|---|---|---|
| Sodium cocosulfate | 20 AM | 20 AM | 20 AM |
| Syndopal 300 (Stephenson)* | 73.5** | 73.5 | 73.5 |
| Hydroxypropyl guar hydroxypropyl | 2.5 AM | — | — |
| trimonium chloride | | | |
| Polyquaternium 10 | — | 2 AM | — |
| Guar hydroxypropyl trimonium | — | — | 2 AM |
| chloride | | | |
| Caprylic/capric triglyceride | 2 AM | 2 AM | 2 AM |
| Fragrance | 2 AM | 2 AM | 2 AM |
| Water | qs 100 | qs 100 | qs 100 |

*Syndopal 300: mixture of sodium cocoyl isethionate (25-50%), hydrogenated plant oil (25-50%), polyglyceryl-4 laurate (5-10%), glycerol (1-5%), water (5-10%)
**amount of commercial product
AM = active material

TABLE 2

| Compositions | D | E | F |
|---|---|---|---|
| Sodium cocosulfate | 20 AM | 20 AM | 20 AM |
| Syndopal 300 (Stephenson) | 73.5** | 73.5 | 73.5 |
| Hydroxypropyl guar hydroxypropyl | 1 AM | — | — |
| trimonium chloride | | | |
| Polyquaternium 7 | — | 0.4 AM | — |
| Polyquaternium-6 | — | 1 AM | — |
| Guar hydroxypropyl trimonium | 1.5 AM | — | 1 AM |
| chloride | | | |
| Selenium sulfide | — | — | 1 AM |
| Undecane (and) tridecane (70/30) | — | — | 1 AM |
| Caprylic/capric triglyceride | 2 AM | 2 AM | 1 AM |
| Fragrance | 2 AM | 2 AM | 2 AM |
| Water | qs 100 | qs 100 | qs 100 |

TABLE 3

| Compositions | G | H | I | J |
|---|---|---|---|---|
| Sodium cocosulfate | 20 AM | 20 AM | 20 AM | 20 AM |
| Syndopal 300 (Stephenson)* | 73.5** | 73.5 | 73.5 | 73.5 |
| Polyquaternium 67 | 1.6 | — | — | — |
| Guar hydroxypropyl trimonium | — | 1 AM | 1 AM | 1 AM |
| chloride | | | | |
| Dicaprylyl carbonate | — | 2 AM | — | — |
| Squalane | — | — | 2 AM | — |
| Diisopropyl adipate | — | — | — | 2 AM |
| Caprylic/capric triglyceride | 2 AM | — | — | — |
| Fragrance | 2 AM | 2 AM | 2 AM | 2 AM |
| Water | qs 100 | qs 100 | qs 100 | qs 100 |

The compositions are prepared by extrusion and are in the form of bars that are easy to hold in the hand.

Method of application: the bar can be applied directly to wet hair in order to generate the foam, and then the foam can be redistributed as with a usual liquid shampoo.

It is also possible to generate the foam in the hand with water, and then to redistribute the foam on the hair.

The composition can be left on for a few moments, then the hair is rinsed with water.

The compositions make it possible to generate an abundant and creamy foam that is easy to distribute over the entire head of hair. The foam initiation (obtaining of the foam) is particularly rapid.

After massaging, the compositions are very easy to rinse off. The wet hair is subsequently easy to disentangle and is smooth to the touch. After drying, the hair is manageable, in individual strands, and soft.

1. Solid composition comprising:
a) one or more anionic surfactants of sulfate type, present in a total amount ranging from 10% to 35% by weight, relative to the total weight of the composition;
b) one or more anionic surfactants of sulfonate type;
c) one or more cationic polymers;
d) one or more (poly)glycerol esters.

2. Composition according to claim 1, characterized in that the anionic surfactant(s) of sulfate type are chosen from alkyl sulfates, alkyl ether sulfates, alkylamido sulfates, alkylamido ether sulfates, alkylaryl polyether sulfates, monoglyceride sulfates; and salts thereof and mixtures thereof, the alkyl groups of these compounds comprising in particular from 8 to 30 carbon atoms, the aryl group preferably denoting a phenyl or benzyl group; it being possible for these compounds to be polyoxyalkylenated, and then preferably comprising from 1 to 50 ethylene oxide units.

3. Composition according to claim 1, characterized in that the anionic surfactant(s) of sulfate type are chosen from:
alkyl sulfates, in particular C₈ to C₂₆, and preferably C₁₀ to C₂₂, alkyl sulfates;
alkyl ether sulfates, in particular C₈ to C₂₆, and preferably C₁₀ to C₂₂, alkyl ether sulfates, preferably comprising from 2 to 10 ethylene oxide units;
in particular in the form of alkali metal, alkaline earth metal, ammonium or amino alcohol salts, and
mixtures thereof.

4. Composition according to claim 1, characterized in that the total amount of the anionic surfactant(s) of sulfate type ranges from 10% to 30% by weight, more preferentially from 10% to 25% by weight, even better still from 15% to 25% by weight, relative to the total weight of the composition.

5. Composition according to claim 1 characterized in that the anionic surfactant(s) of sulfonate type are chosen from alkyl sulfonates, alkylamide sulfonates, alkylaryl sulfonates, alpha-olefin sulfonates, paraffin sulfonates, alkylsulfosuccinates, alkyl ether sulfosuccinates, alkylamide sulfosuccinates, alkyl sulfoacetates, N-acyltaurates, acylisethionates, and also salts thereof and mixtures thereof, the alkyl groups of these compounds comprising from 8 to 30 carbon atoms, preferably from 8 to 26 and more preferentially from 10 to 22 carbon atoms; the aryl group preferably denoting a phenyl or benzyl group; it being possible for these compounds to be polyoxyalkylenated, in particular polyoxyethylenated and then preferably comprising from 1 to 50 ethylene oxide units, and more preferentially from 2 to 10 ethylene oxide units.

6. Composition according to claim 1, characterized in that the anionic surfactant(s) of sulfonate type are chosen from the compounds of formula (I) below:
R₁—COX—R₂—SO₃M (I),
in which:
R₁ represents a linear or branched, preferably linear, alkyl group comprising from 8 to 30 carbon atoms, preferably from 8 to 26 carbon atoms, and more preferentially from 10 to 22 carbon atoms;
X represents an oxygen atom or an —N(CH₃)— or —NH— group, preferably an oxygen atom;
R₂ represents a linear or branched alkyl group comprising from 1 to 4 carbon atoms; and
M denotes a hydrogen atom, an ammonium ion, an ion derived from an alkali metal or an alkaline-earth metal or an ion derived from an organic amine.

7. Composition according to claim 1, characterized in that the total amount of the anionic surfactant(s) of sulfonate type ranges from 5% to 45% by weight, preferably from 10% to 40% by weight, more preferentially from 20% to 35% by weight, even better still from 25% to 35% by weight, relative to the total weight of the composition.

8. Composition according to claim 1, characterized in that the cationic polymers are chosen from associative cationic polymers and non-associative cationic polymers, and mixtures thereof.

9. Composition according to claim 1, characterized in that the cationic polymer(s) are non-associative and are chosen from:
(1) homopolymers or copolymers derived from acrylic or methacrylic esters or amides and including at least one of the units having the following formula: 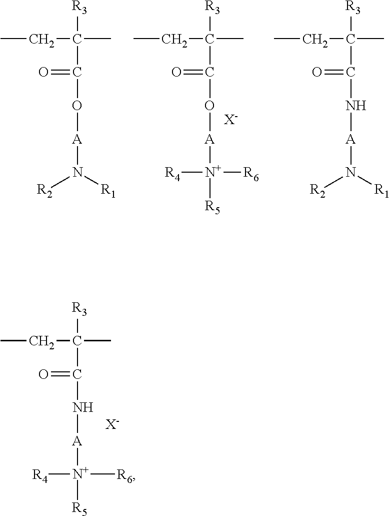
in which:
R₃, which may be identical or different, denote a hydrogen atom or a CH₃ radical;
A, which may be identical or different, represent a linear or branched divalent alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group having from 1 to 18 carbon atoms or a benzyl radical;
R₁ and R₂, which may be identical or different, represent a hydrogen atom or an alkyl group having from 1 to 6 carbon atoms; and
X denotes an anion derived from a mineral or organic acid, such as a methosulfate anion or a halide such as chloride or bromide;
(2) cationic polysaccharides;
(3) polymers constituted of piperazinyl units and divalent alkylene or hydroxyalkylene radicals containing linear or branched chains, optionally interrupted with oxygen, sulfur or nitrogen atoms or with aromatic or heterocyclic rings, and also the oxidation and/or quaternization products of these polymers;
(4) water-soluble polyaminoamides prepared in particular by polycondensation of an acidic compound with a polyamine;
(5) polyamino amide derivatives resulting from the condensation of polyalkylene polyamines with polycarboxylic acids followed by alkylation with difunctional agents;
(6) polymers obtained by reacting a polyalkylene polyamine including two primary amine groups and at least one secondary amine group with a dicarboxylic acid chosen from diglycolic acid and saturated aliphatic dicarboxylic acids containing from 3 to 8 carbon atoms;
(7) alkyldiallylamine or dialkyldiallylammonium cyclopolymers;
(8) diquaternary ammonium polymers comprising repeating units of formula (VI): 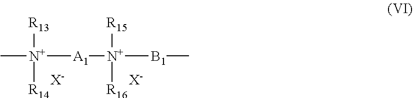
in which:
R₁₃, R₁₄, R₁₅ and R₁₆, which may be identical or different, represent aliphatic, alicyclic or arylaliphatic radicals comprising from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally comprising a second heteroatom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;
A₁ and B₁ represent divalent polymethylene groups comprising from 2 to 20 carbon atoms which may be linear or branched, and saturated or unsaturated, and which may contain, linked to or inserted in the main chain, one or more aromatic rings, or one or more oxygen or sulfur atoms or sulfoxide, sulfone, disulfide, amino, alkylamino, hydroxyl, quaternary ammonium, ureido, amide or ester groups, and
X⁻ denotes an anion derived from a mineral or organic acid;
it being understood that A₁, R₁₃ and R₁₅ can form, with the two nitrogen atoms to which they are attached, a piperazine ring;
in addition, if A₁ denotes a saturated or unsaturated and linear or branched alkylene or hydroxyalkylene radical, B₁ can also denote a (CH₂)ₙ—CO-D-OC—(CH₂)ₙ— group in which D denotes:
a) a glycol residue of formula —O—Z—O—, in which Z denotes a linear or branched hydrocarbon-based radical or a group corresponding to one of the following formulae: —(CH₂—CH₂—O)ₓ—CH₂—CH₂— and —[CH₂—CH(CH₃)—O]_{y}—CH₂—CH(CH₃)—, where x and y denote an integer from 1 to 4, representing a defined and unique degree of polymerization or any number from 1 to 4 representing an average degree of polymerization;
b) a bis-secondary diamine residue, such as a piperazine derivative;
c) a bis-primary diamine residue of formula: —NH—Y—NH—, in which Y denotes a linear or branched hydrocarbon-based radical, or else the divalent radical —CH₂—CH₂—S—S—CH₂—CH₂—; or
d) a ureylene group of formula: —NH—CO—NH—;
(9) polyquaternary ammonium polymers comprising units of formula (VIII): 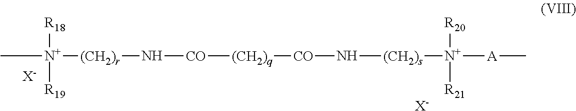
in which:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH radical, where p is equal to 0 or to an integer of between 1 and 6, with the proviso that R₁₈, R₁₉, R₂₀ and R₂₁ do not simultaneously represent a hydrogen atom;
r and s, which may be identical or different, are integers between 1 and 6;
q is equal to 0 or to an integer between 1 and 34;
X⁻ denotes an anion, such as a halide, and
A denotes a dihalide radical or preferably represents —CH₂—CH₂—O—CH₂—CH₂—;
(10) quaternary polymers of vinylpyrrolidone and of vinylimidazole;
(11) polyamines; and
(12) polymers including in their structure:
(a) one or more units corresponding to formula (A) below: 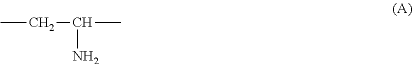
(b) optionally one or more units corresponding to formula (B) below:

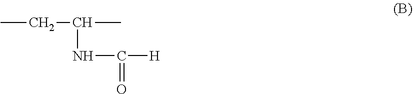

10. Composition according to claim 1, characterized in that the cationic polymers are associative and are chosen from quaternized (poly)hydroxyethylcelluloses modified with groups comprising at least one fatty chain, cationic polyvinyllactam polymers, and mixtures thereof, more preferentially from quaternized (poly)hydroxyethylcelluloses modified with groups including at least one alkyl group containing at least 10 carbon atoms, preferentially ranging from 10 to 22 carbon atoms and more preferentially ranging from 12 to 16 carbon atoms, and mixtures thereof.

11. Composition according to claim 1, characterized in that the cationic polymer(s) are chosen from non-associative celluloses, cationic galactomannan gums, quaternized (poly)hydroxyethylcelluloses modified with groups comprising at least one alkyl group containing at least 10 carbon atoms, preferentially ranging from 10 to 22 carbon atoms, and more preferentially ranging from 12 to 16 carbon atoms, and mixtures thereof, even more preferentially from cellulose ether derivatives comprising quaternary ammonium groups, cationic guar gums, quaternized (poly)hydroxyethylcelluloses modified with groups comprising at least one alkyl group containing at least 10 carbon atoms, preferentially ranging from 10 to 22 carbon atoms, and more preferentially ranging from 12 to 16 carbon atoms, and mixtures thereof.

12. Composition according to claim 1, characterized in that the total amount of the cationic polymer(s) ranges from 0.05% to 20% by weight, preferably from 0.1% to 10% by weight, and more preferentially from 0.5% to 8% by weight, even better still from 1% to 5% by weight, even better still from 1% to 3% by weight, or even from 2% to 3% by weight, relative to the total weight of the composition.

13. Composition according to claim 1, characterized in that the (poly)glycerol ester(s) are chosen from:
monoesters or polyesters of linear or branched C₈ to C₄₀ acids, which are monoglycerolated or polyglycerolated, comprising from 1 to 50 mol of glycerol, preferably from 1 to 20 or even from 2 to 10 mol of glycerol; in particular monoesters or diesters of linear or branched C₈ to C₃₂, better still C₁₀ to C₂₈ or even C₁₀ to C₂₄ acids, comprising from 1 to 50 mol of glycerol, preferably from 1 to 20 or even from 2 to 10 mol of glycerol, and more preferentially monoesters or diesters of linear or branched C₈ to C₄₀, better still C₈ to C₃₂, even better still C₁₀ to C₂₈, or even C₁₀ to C₂₄, acids, which are monoglycerolated or polyglycerolated, comprising from 1 to 50 mol of glycerol, preferably from 1 to 20 or even from 2 to 10 mol of glycerol;
triglycerides constituted of esters of fatty acids and glycerol, the fatty acids of which may be C₆ to C₂₄, preferably C₈-C₂₂, fatty acids;
mixtures thereof.

14. Composition according to claim 1, characterized in that the total amount of the (poly)glycerol ester(s) ranges from 0.1% to 60% by weight, preferably from 1% to 50% by weight, more preferentially from 3% to 45% by weight, even more preferentially from 5% to 40% by weight, better still from 5% to 30% by weight, even better still from 5% to 20% by weight, relative to the total weight of the composition, relative to the total weight of the composition.

15. Composition according to claim 1, characterized in that it also comprises one or more polyols preferably chosen from sorbitol, glycerol, propylene glycol, and mixtures thereof, more preferentially from glycerol.

16. Composition according to claim 15, characterized in that the total amount of the polyol(s) ranges from 0.1% to 20% by weight, preferably from 0.5% to 15% by weight, more preferentially from 1% to 8% by weight, even better still from 1% to 5% by weight, relative to the total weight of the composition.

17. Cosmetic process for treating keratin fibres, in particular human keratin fibres such as the hair, comprising the application to said keratin fibres of a solid composition as defined in claim 1, the solid composition being applied directly to said keratin fibres or after having been wetted beforehand with water.

18. Use of a solid composition as defined in claim 1, for washing and/or conditioning keratin fibres, in particular human keratin fibres such as the hair.
